(19) United States
(12) Patent Application Publication
(10) Pub. No.: US 2009/0069678 A1
(43) Pub. Date: Mar. 12, 2009

(54) Method and Apparatus for Ultrasonic Drug Delivery and Medical Diagnostic Imaging Apparatus

(75) Inventors: Yoshiaki Taniyama, Suita-shi (JP); Ryuichi Morishita, Suita-shi (JP); Takashi Miyake, Suita-shi (JP); Katsuhiko Fujimoto, Ohtawara-shi (JP)

Correspondence Address: WENDEROTH, LIND & PONACK, L.L.P., 2033 K STREET N. W., SUITE 800, WASHINGTON, DC, 20006-1021, US

(73) Assignee: OSAKA UNIVERSITY, Suita-shi (JP)

(21) Appl. No.: 12/087,407

(22) PCT Filed: Jan. 5, 2007
(86) PCT No.: PCT/JP2007/050032

§ 371 Date: Aug. 25, 2008

(30) Foreign Application Priority Data
Jan. 6, 2006 (JP) 2006-001821

(51) Int. Cl. A61N 7/00; A61B 8/00

(52) U.S. Cl. 600/439; 604/22

(57) ABSTRACT

According to the present invention, there are provided an ultrasonic drug delivery method and an ultrasonic drug delivery apparatus each capable of performing more localized and efficient drug-delivery with the aid of ultrasonic irradiation under static pressure, which increases the effect of drug delivery to deep tissue parts in treatment by ultrasonic irradiation to a living body for delivery of drugs such as nucleic acids (such as DNA, RNA, decoys, and RNAi), proteins and pharmaceutical compounds, and there is also provided a medical diagnostic imaging apparatus.

### TECHNICAL FIELD

The present invention relates to an ultrasonic drug delivery method and apparatus for delivering drugs such as nucleic acids, proteins and pharmaceutical compounds into cells, nuclei, tissues, and so on under ultrasonic irradiation to a subject such as a patient, and also relates to a medical diagnostic imaging apparatus using such an ultrasonic drug delivery apparatus.

### BACKGROUND ART

In recent years, attention has been given in every medical field to therapeutic methods capable of achieving a radical remedy at a very early stage, such as MIT (Minimally Invasive Treatment), a gene therapy and a regenerative medical technique. For example, arteriosclerosis- or thrombosis-induced diseases such as ischemic brain diseases and ischemic heart diseases have a high recurrence rate, which is a big problem. In Japan, the number of hyperlipidemic patients has also increased as eating habits have been westernized. Thus, attention has been given to a gene transfer therapy which includes suppressing local recurrence or regenerating blood vessels in tissues with complete infarction so that ischemia can be ameliorated.

Concerning the angiogenesis factor, for example, gene therapies that promote angiogenesis to treat diabetic limb ischemia or necrosis are actually performed in Western countries and provide benefits. Concerning angiogenesis inhibitory factors having the contrary function, it is known that metabolically active tumor cells produce signals to require angiogenesis and thus proliferate. Therefore, angiogenesis inhibitory factors may be used to suppress tumor growth, when regeneration of nutrient vessels is suppressed by delivery of an angiogenesis factor.

The major gene therapy approaches are viral vector-based methods such as methods that include integrating a target gene into a retrovirus with reduced toxicity and introducing the gene into a gene of a target cell by infection, because such methods have high delivery efficiency. Recently in Western countries, however, death has been caused by viral toxicity itself in some gene therapies, and therefore, some hesitation in using virus-based gene delivery has occurred in Japan and other countries. In view of such circumstances, other gene delivery techniques have been investigated.

Examples of non-viral vector methods include chemical methods using liposomes or the like and delivery methods using microinjection, gene gun, electroporation, laser, or the like. In recent years, attention has been given to an ultrasonic sonoporation-assisted gene delivery technique, which is one of new delivery techniques.

This ultrasonic gene delivery technique is based on a phenomenon in which when an ultrasonic contrast medium (bubbles) used for diagnostic imaging collapses upon ultrasonic irradiation, a microjet is generated and to form a temporary pore in a cell membrane (sonoporation phenomenon). Genes, proteins, or the like are directly introduced into a cell or a nucleus through the pore.

In general, continuous irradiation of an ultrasonic wave causes the generation of minute bubbles called cavitation, which also induces a similar phenomenon. Concerning ultrasonic gene delivery techniques, methods in combination with artificial injecting bubbles (a contrast medium) to increase the efficiency can further enhance the delivery efficiency and have been generally known. For example, such ultrasonic gene delivery techniques are disclosed in the following documents:

### JP-A NOS. 9-502191, 2001-507207, 2001-512329 and 2004-261253;

### (a) Hiroshi FURUHATA and Yoshinobu MANOBE, “Choonpa Idenshi Donyu no Tenkai (Development of Ultrasonic Gene Delivery),” BME, Japanese Society for Medical and Biological Engineering, Jul. 10, 2002, Vol. 16, No. 7, pp. 3-7;

(b) Yoshiaki TABUCHI and Takashi KONDO, “Choonpa Yudo Idenshi Chiryo (Ultrasonic Wave-Induced Gene Therapy),” a separate volume of Igaku No Ayumi (Medical Progress), “Choonpa Igaku Saizensen (The Front of Ultrasonic Medical Science),” published by Ishiyaku Pub. Inc., pp. 203-208, 2004; and
(c) Katsuhiko FUJIMOTO and Takehide ASANO, “Shusoku-Choonpa niyoru Chiryoho to Mondaiten (Therapeutic Methods and Problems with Focused Ultrasonic Wave),” a separate volume of Igaku No Ayumi (Medical Progress), “Choonpa Igaku Saizensen (The Front of Ultrasonic Medical Science),” published by Ishiyaku Pub. Inc., pp. 198-202, 2004.

In order to enhance drug delivery effects, ultrasonic gene delivery techniques are used in combination with an ultrasonic contrast medium such as Levovist, which has been approved as a diagnostic contrast medium for clinical trials and used for ultrasonic diagnostic image-based observation of hemodynamics, perfusion and the like in tissues, and Optison (not approved yet in Japan). Such techniques have the potential to safely deliver drugs and therefore receive attention.

Now, there is the increasing use of echography techniques in which ultrasonic diagnosis is performed with the aid of an ultrasonic contrast medium (microbubbles). This ultrasonic diagnosis is very compatible with the ultrasonic therapy described above, and they are easy to be combined. Therefore, they are very useful for heating therapy using high intensity focused ultrasound (HIFU) or for monitoring ultrasonic therapy with an ultrasonic lithotripsy system or the like. For example, these techniques are disclosed in the following documents: JP-A NOS. 6-78930, 11-226046 and

Katsuhiko FUJIMOTO and Takehide ASANO, “Shusoku-Choonpa niyoru Chiryoho to Mondaiten (Therapeutic Methods and Problems with Focused Ultrasonic Wave),” a separate volume of Igaku No Ayumi (Medical Progress), “Choonpa Igaku Saizensen (The Front of Ultrasonic Medical Science),” published by Ishiyaku Pub. Inc., pp. 198-202, 2004.

As genetic analysis progresses, the idea of molecular imaging has been quickly and widely applied to medical diagnostic imaging, which has previously undergone dramatic progress with respect to morphological diagnosis. Molecular imaging may be broadly divided into: literal molecular imaging by which molecules themselves in the order of nanometers are imaged using light or X-rays; and functional imaging by which the uptake of drugs into molecules or drug metabolism is imaged so that the behavior of molecules is indirectly imaged. Examples of the former include imaging with a fluorescence microscope or an X-ray microscope, and examples of the latter include imaging with a nuclear medicine device (such as a PET or SPECT device) or MRS.

The former is mainly used in laboratories, because it has a problem with the imaging-energy penetration depth in tissues and the problem of exposure to radiations. In contrast, the latter has been recently applied in a wide clinical field, because in the latter technique, the imaging of metabolic function can be enhanced using a combination of a radionuclide of a labeled target molecule and a contrast medium, though the resolution is relatively low. In particular, attention has been recently given to a new application such as PET-CT, in which PET with relatively low resolution is complemented by CT with relatively high morphological resolution so that metabolic information can be superimposed on three-dimensional morphological images being displayed.

These molecular imaging techniques enable imaging of metabolically active tumor cells in contrast to normal tissues and would enable imaging of the expression of a specific gene and the production of proteins in the future. Therefore, molecular imaging can provide useful information that is directly applicable to treatment planning, very early diagnosis, and monitoring of gene therapy or the like.

The prevention of recurrence and rejection, which are problems in vascular transplantation for coronary artery diseases and transplantation of organs such as kidney, is a very important object of transplantation therapy. To date, however, there has been no system capable of effectively delivering immunosuppressive agents or immune function suppression genes to organs to be transplanted.

The delivery efficiency is also still lower in conventional ultrasonic gene delivery techniques than in viral vector-based methods. Ultrasonic delivery has been effective in delivering drugs to the organ or tissue surface capable of being in contact with drugs, because it is based on the sonoporation phenomenon caused by a microjet upon collapse of microbubbles. However, delivery to local deep portions has been quite difficult for ultrasonic delivery.

### DISCLOSURE OF THE INVENTION

### Problems to be Solved by the Invention

An object of the present invention is to provide an ultrasonic drug delivery method and an ultrasonic drug delivery apparatus each capable of facilitating more localized and efficient drug-delivery with the aid of ultrasonic irradiation under static pressure, which increases the effect of drug delivery to deep tissue parts in treatment by ultrasonic irradiation to a living body for delivery of drugs such as nucleic acids (such as DNA, RNA, decoys, and RNAi), proteins, and pharmaceutical compounds (hereinafter, these are generically referred to as “drug” or “drugs”) and to provide a medical diagnostic imaging apparatus.

### Means for Solving the Problems

The invention is directed to:

(1) an ultrasonic drug delivery method, including: applying a static pressure to a subject, while applying an ultrasonic wave to the subject, in order to deliver a drug to the subject;

(2) the ultrasonic drug delivery method according to Item (1), wherein the static pressure has a constant positive value;

(3) the ultrasonic drug delivery method according to Item (1), wherein the static pressure is from 1.05 to 3 atmospheres;

(4) the ultrasonic drug delivery method according to Item (1), wherein the ultrasonic wave has a continuous wave;

(5) the ultrasonic drug delivery method according to Item (1), wherein the ultrasonic wave has a frequency of 100 kHz to 10 MHz;

(6) an ultrasonic drug delivery apparatus, including: a static pressure application unit that applies a static pressure to a subject; and an ultrasonic wave application unit that applies an ultrasonic wave to the subject, wherein a drug is delivered to the subject, while the static pressure and the ultrasonic wave are applied to the subject;

(7) the ultrasonic drug delivery apparatus according to Item (6), wherein the static pressure application unit applies the static pressure having a constant positive value to the subject;

(8) the ultrasonic drug delivery apparatus according to Item (6), wherein the static pressure application unit applies the static pressure of 1.05 to 3 atmospheres to the subject;

(9) the ultrasonic drug delivery apparatus according to Item (6), wherein the static pressure application unit includes a pressure container that accommodates the subject, a pressurizing mechanism that pressurizes the inside of the pressure container so as to apply the static pressure to the subject, and a pressure sensor that detects the pressure applied to the inside of the pressure container;

(10) the ultrasonic drug delivery apparatus according to Item (9), wherein the pressurizing mechanism automatically or manually pressurizes the inside of the pressure container to the static pressure;

(11) the ultrasonic drug delivery apparatus according to Item (9), wherein the pressurizing mechanism includes a pressure pump or a syringe pressurizer;

(12) the ultrasonic drug delivery apparatus according to Item (6), wherein the ultrasonic wave application unit emits the ultrasonic wave in the form of a continuous wave;

(13) the ultrasonic drug delivery apparatus according to Item (6), wherein the ultrasonic wave application unit applies, to the subject, the ultrasonic wave with a frequency of 100 kHz to 10 MHz;

(14) the ultrasonic drug delivery apparatus according to Item (6), wherein the ultrasonic wave application unit includes at least one ultrasonic transducer that emits the ultrasonic wave;

(15) the ultrasonic drug delivery apparatus according to Item (9), further including a drive unit that is provided outside the pressure container to drive the at least one ultrasonic transducer, and an airtight cable that connects the drive unit to the pressure container;

(16) the ultrasonic drug delivery apparatus according to Item (6), wherein the pressure container is a standard container for irradiation of the ultrasonic wave to the subject, the ultrasonic wave application unit has an ultrasonic transducer to emit the ultrasonic wave and accommodates an acoustic medium that is provided with the ultrasonic transducer and acoustically connects the ultrasonic transducer to the standard container, and the ultrasonic wave application unit has a holding part that holds the standard container such that the standard container coincides with a region on which the irradiation of the ultrasonic wave emitted from the ultrasonic transducer is focused;

(17) the ultrasonic drug delivery apparatus according to Item (6), wherein the pressure container is cylindrical, the ultrasonic wave application unit includes a plurality of ultrasonic transducers to emit the ultrasonic wave, and the plurality of ultrasonic transducers are arranged at least on a cylindrical inner wall of the pressure container;

(18) the ultrasonic drug delivery apparatus according to Item (17), wherein the plurality of ultrasonic transducers each emit the ultrasonic wave such that the ultrasonic wave is uniformly irradiated throughout the subject;

(19) the ultrasonic drug delivery apparatus according to Item (17), further including a drive control unit that controls the drive of the plurality of ultrasonic transducers by at least phase control;

(20) the ultrasonic drug delivery apparatus according to Item (6), wherein the pressure container is a standard container for irradiation of the ultrasonic wave to the subject, the pressurizing mechanism is a syringe pressurizer and pressurizes the inside of the standard container such that the static pressure is applied to the subject, and the ultrasonic wave application unit includes an ultrasonic transducer to emit the ultrasonic wave and applies the ultrasonic wave from the outside of the standard container to the subject;

(21) the ultrasonic drug delivery apparatus according to Item (6), wherein the pressure container is made of a material that allows delivery of the drug to the subject to be checked with a molecular imaging device;

(22) the ultrasonic drug delivery apparatus according to Item (21), wherein the material of which the pressure container is made includes an optically-transparent material that allows fluorescent imaging;

(23) the ultrasonic drug delivery apparatus according to Item (21), wherein the material of which the pressure container is made is transparent to radioactive rays or X-rays;

(24) the ultrasonic drug delivery apparatus according to Item (21), wherein the material of which the pressure container is made allows magnetic resonance imaging;

(25) a medical diagnostic imaging apparatus including the ultrasonic drug delivery apparatus according to any one of Items (6) to (24);

(26) the medical diagnostic imaging apparatus according to Item (25), further including an ultrasonic transducer that is for producing an ultrasonic image of the subject and independently placed in the pressure container, wherein the ultrasonic image of the subject is output and displayed, while the drug is delivered to the subject under application of the static pressure and the ultrasonic wave;

(27) the medical diagnostic imaging apparatus according to Item (25), wherein the ultrasonic transducer for producing the ultrasonic image of the subject also serves to apply the ultrasonic wave to the subject for delivery of the drug to the subject; and

(28) the medical diagnostic imaging apparatus according to Item (25), further including a device for positron emission tomography (PET), magnetic resonance imaging (MRI) or X-ray computed tomography (CT).

### EFFECT OF THE INVENTION

The ultrasonic drug delivery method, the ultrasonic drug delivery apparatus and the medical diagnostic imaging apparatus provided according to the present invention use ultrasonic irradiation under static pressure, which increases the effect of drug delivery to deep tissue parts in treatment by ultrasonic irradiation to a living body and by delivery of drugs so that they can achieve more localized and efficient delivery of drugs.

### BRIEF DESCRIPTION OF DRAWINGS

FIG. 1 is a schematic diagram showing the whole of a medical diagnostic imaging apparatus including an ultrasonic drug delivery apparatus according to a first embodiment of the present invention.

FIG. 2 is a diagram showing a small vessel held in an applicator of the apparatus.

FIG. 3 is a graph showing degrees of depth of delivery to vascular tissues in the presence and absence of static pressure applied from the apparatus.

FIG. 4 is a schematic diagram showing an ultrasonic drug delivery apparatus according to a second embodiment of the invention.

FIG. 5 is a diagram showing a focal region of an ultrasonic wave applied to a subject in an airtight pressure container.

FIG. 6 is a schematic diagram showing an ultrasonic drug delivery apparatus according to a third embodiment of the present invention.

### DESCRIPTION OF THE REFERENCE NUMERALS

In the drawings, reference numeral 1 represents an airtight pressure container, 2 amount, 3 a small vessel, 4 an applicator, a solution, 6 a subject (a sample to which delivery is to be made), 7 a pressure cap, 8 a housing, 9 an ultrasonic transducer, 10 water, 11 a pressure tube, 12 a pressure pump, 13 a pressure sensor, 14 a water supply circuit, 15 a water supply piping, 16 a valve, 17 a driver, 18 a controller, 19 a medical diagnostic imaging apparatus, 20 a display, 21 an input device, 30 an airtight pressure container, 31 a cover, 32 ultrasonic transducers (a group of ultrasonic transducers), 33 a solution, 34 a subject, 35 a pressure tube, 36 a pressure pump, 37 a pressure sensor, 38 a driver, 39 a controller, 40 a subject, 41 a syringe pressurizer, 42 a pressure tube, 43 a pressure chamber, 44 a cylinder, and 45 a controller.

### BEST MODE FOR CARRYING OUT THE INVENTION

A first embodiment of the present invention will be described below with reference to the drawings.

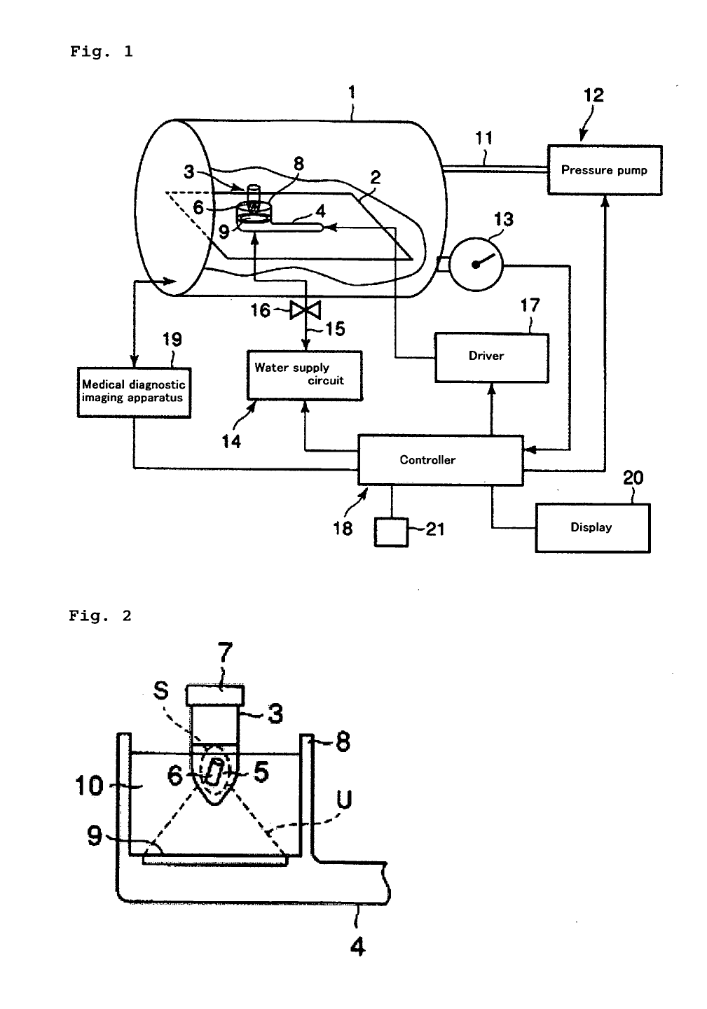

FIG. 1 is a schematic diagram showing the whole of a medical diagnosis imaging apparatus including an ultrasonic drug delivery apparatus. A mount 2 is placed in an airtight pressure container 1. The inside of the airtight pressure container 1 is kept at a static pressure. When a molecular imaging device is a PET system or any other fluorescent imager, the airtight pressure container 1 should be made of an optically-transparent material that allows fluorescent imaging. When a molecular imaging diagnosis device is a nuclear medicine device, an X-ray device, an optical device, an MRI system, or the like, the airtight pressure container 1 should be made of a material transparent to radioactive rays or X-rays. When the airtight pressure container 1 is made of a material that allows drug delivery to the target region of a subject 6 to be checked with a molecular imaging diagnosis device, namely when a material suitable for the molecular imaging diagnosis device is selected, the drug delivery efficiency can be reliably monitored in the process of the drug delivery.

An applicator 4 serving as a holder to hold a small vessel 3 such as a standard container is placed on the mount 2. The standard container is normally used for in vitro experiments and the like and typically includes a 15 ml tube (manufactured by Greiner). For example, the small vessel 3 is made of a resin such as a plastic resin.

FIG. 2 shows the small vessel 3 held in the applicator 4. The small vessel 3 contains a solution 5, for example, which includes a cell suspension and microbubbles mixed therein. A subject (a sample to which delivery is to be made) 6 such as an isolated organ for transplantation or a small animal is immersed in the solution 5. For example, the subject 6 in the small vessel 3 may also be an isolated organ or small animal to which a drug containing microbubbles has been administered. The small vessel 3 is sealed with a pressure cap 7 so that the inside of the small vessel 3 is kept in an airtight state.

The applicator 4 has a housing 8. An ultrasonic transducer 9 is placed on the bottom of the housing 8. For example, the ultrasonic transducer 9 is a spherical shell-shaped sound source of a sound collecting type. An ultrasonic wave U with a frequency of 100 kHz to 10 MHz is emitted from the ultrasonic transducer 9 and focused on a focal region S. The housing 8 is typically filled with water 10 or any other ultrasonic medium such as Sono Jelly. Alternatively, a water-filled vessel or a water bag may be placed as an ultrasonic medium on the front side of the ultrasonic transducer 9.

The small vessel 3 is held in the housing 8 such that the lower part of the vessel 3 where the subject 6 is held and immersed in the solution 5 is immersed in water 10 and that the subject 6 is placed in the focal region S of the ultrasonic wave U emitted from the ultrasonic transducer 9, namely such that the subject 6 is placed on a plane where the energy of the ultrasonic wave U emitted from the ultrasonic transducer 9 is irradiated.

A pressure pump 12 is connected to the airtight pressure container 1 through a pressure tube 11. The pressure pump 12 placed outside the airtight pressure container 1 injects a gas such as oxygen or air into the airtight pressure container 1 so that the pressure in the airtight pressure container 1 can be controlled. The airtight pressure container 1 is provided with an openable cover so that the applicator 4 holding the small vessel 3 can be set in or removed from the airtight pressure container 1.

A pressure sensor 13 is attached to the airtight pressure container 1. The pressure sensor 13 placed outside the airtight pressure container 1 detects the pressure in the airtight pressure container 1 and outputs a pressure detection signal.

A water supply circuit 14 is provided outside the airtight pressure container 1 and connected through a water supply piping 15 to the housing 8 or a water bag in the airtight pressure container 1. The water supply circuit 14 supplies water 10 through the water supply piping 15 into the housing 8 or the water bag so that the housing 8 or the water bag is filled with water 10. A valve 16 is connected to the water supply piping 15. The valve 16 prevents backflow of water from the housing 8 or the water bag to the water supply circuit 14.

A driver 17 provided outside the airtight pressure container 1 outputs a drive signal to the ultrasonic transducer 9 to drive the ultrasonic transducer 9, for example, at a frequency of 100 kHz to 10 MHz, so that the ultrasonic wave U is generated. A cable that maintains the airtight structure of the airtight pressure container 1 (hereinafter referred to as “airtight cable”) is used to connect between the driver 17 and the ultrasonic transducer 9 and to send the drive signal output from the driver 17 to the ultrasonic transducer 9.

A controller 18 outputs a drive signal to the pressure pump 12 to drive the pressure pump 12. The controller 18 also inputs the pressure detection signal output from the pressure sensor 13 to control the pressure in the airtight pressure container 1 at a constant positive static pressure, for example, a specific static pressure in the range of 1.05 to 3 atmospheres.

While the controller 18 keeps the inside of the airtight pressure container 1 at a static pressure, it sends a drive control signal to the driver 17 so that the ultrasonic transducer 9 is driven typically at a frequency of 100 kHz to 10 MHz to generate the ultrasonic wave U.

The controller 18 sends opening and closing control signals to the valve 16 to control opening and closing of the valve 16.

A medical diagnostic imaging apparatus 19, a display 20 and an input device 21 are connected to the controller 18. The medical diagnostic imaging apparatus 19 typically includes a molecular imaging device such as a positron emission tomography (PET) device, a fluorescent imager, a nuclear medicine device, an X-ray computed tomography (CT) device, an optical device, or a magnetic resonance imaging (MRI) device (hereinafter, these are generically referred to as “PET or the like”) to produce the PET image, fluorescent image, X-ray CT image, MRI image, or the like (hereinafter, these are generically referred to as “PET image or the like”) of the subject 6. The input device 21 typically includes a mouse and a keyboard.

The controller 18 receives the PET image or the like of the subject 6 transferred from the medical diagnostic imaging apparatus 19 and causes the display 20 to display the diagnostic imaging information of the subject 6 and the state of delivery of a drug into the subject 6. The controller 18 receives operational instructions from the input device 21 and issues, to the driver 17, a command to transmit or stop the ultrasonic wave U from the ultrasonic transducer 9.

Next, a description is given of the operation to facilitate delivery of a drug in the apparatus configured as described above.

The controller 18 sends an opening and closing control signal to the valve 16 to open the valve 16. When the valve 16 is open, the water supply circuit 14 supplies water 10 to the housing 8 or the water bag through the water supply piping 15. When the housing 8 or the water bag is filled with water 10, the controller 18 sends an opening and closing control signal to the valve 16 to close the valve 16. Therefore, backflow of water from the housing 8 or the water bag to the water supply circuit 14 is prevented.

The small vessel 3 contains a solution 5, for example, which includes a cell suspension and microbubbles mixed therein. The subject 6 such as an isolated organ for transplantation or a small animal is immersed in the solution 5. The small vessel 3 is sealed with the pressure cap 7 so that the inside of the small vessel 3 is kept in an airtight state. The small vessel 3 is inserted into the airtight pressure container 1 through an opening which is provided when the cover is opened. The small vessel 3 is held such that the subject 6 is placed in the focal region S of the ultrasonic wave U emitted from the ultrasonic transducer 9, namely such that the subject 6 is placed on a plane where the energy of the ultrasonic wave U emitted from the ultrasonic transducer 9 is irradiated. After the small vessel 3 is put in place, the opening is closed with the cover so that the airtight pressure container 1 is hermetically sealed.

Then, the controller 18 outputs a drive signal to the pressure pump 12 to drive the pressure pump 12. The pressure pump 12 injects a gas such as oxygen or air into the airtight pressure container 1 through the pressure tube 11 to increase the pressure in the airtight pressure container 1. In this process, the pressure sensor 13 detects the pressure in the airtight pressure container 1 and outputs a pressure detection signal.

The controller 18 inputs the pressure detection signal output from the pressure sensor 13 and outputs a drive signal to the pressure pump 12 to keep the inside of the airtight pressure container 1 at a constant positive static pressure, for example, a specific static pressure in the range of 1.05 to 3 atmospheres. The controller 18 causes the display 20 to display the pressure in the airtight pressure container 1 detected by the pressure sensor 13 sequentially.

For example, when the inside of the airtight pressure container 1 is kept at a static pressure of 1.05 atmospheres, the controller 18 sends a control signal to the driver 17 to start the drive. When the driver 17 inputs the drive control signal from the controller 18, it outputs a drive signal to the ultrasonic transducer 9 so that the ultrasonic transducer 9 generates an ultrasonic wave U, for example, with a frequency of 100 kHz to 10 MHz. The lower part of the small vessel 3 where the subject 6 is held and immersed in the solution 5 is immersed in water 10, and the subject 6 is placed on a plane where the energy of the ultrasonic wave U emitted from the ultrasonic transducer 9 is irradiated. Therefore, the ultrasonic wave U emitted from the ultrasonic transducer 9 is irradiated to the subject 6 through the water 10.

The ultrasonic transducer 9 may be automatically driven by the driver 17 that is driven and controlled by the controller 18 or may be driven by manually operating the driver 17.

As described above, the ultrasonic wave U is irradiated to the subject 6, while a static pressure is applied to the subject 6. As a result, the interaction with the microbubbles is facilitated so that delivery of a drug to the subject 6 is facilitated by a microjet that is generated when the microbubbles collapse (the sonoporation phenomenon).

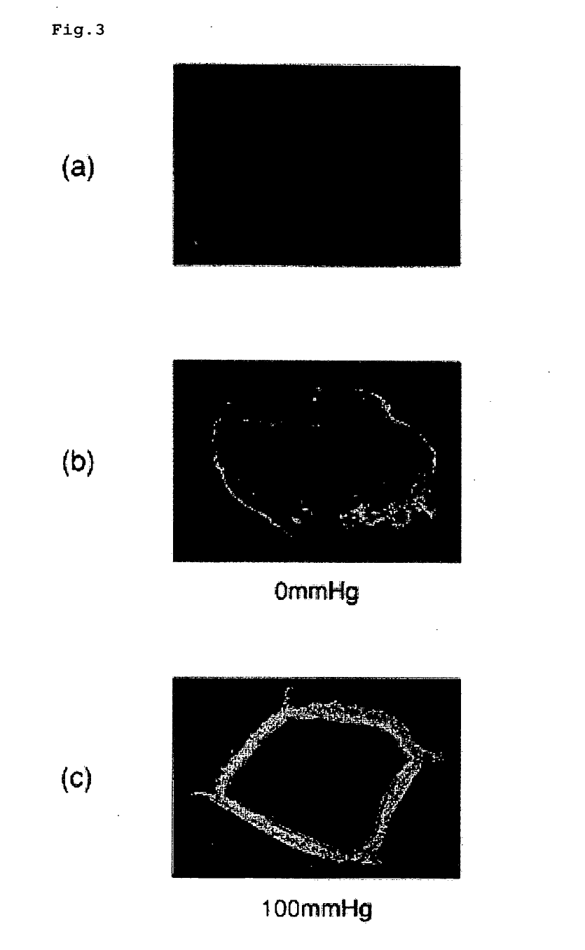

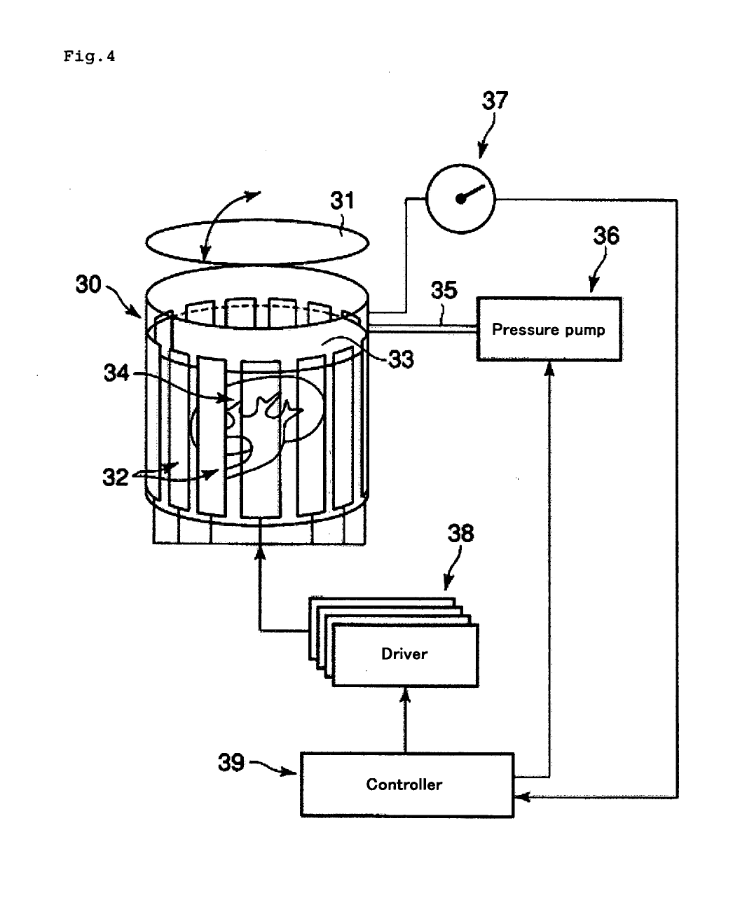

The inventors have conducted basic experiments, which have led to the finding that the delivery to the subject 6 (for example, deep tissue parts) is facilitated under application of static pressure. FIGS. 3(a) to 3(c) show degrees of depth of delivery to vascular tissues in the presence and absence of static pressure. FIG. 3(a) shows a vascular tissue (the subject 6); FIG. 3(b) shows the degree of depth of delivery in the absence of static pressure (0 mmHg); and FIG. 3(c) shows the degree of depth of delivery in the presence of static pressure (100 mmHg). FIG. 3(b) shows that when no pressure is applied, delivery of oligonucleotide is facilitated only in the surface part in contact with the oligonucleotide and bubbles, namely fluorescence is showed only in the surface part. In contrast, FIG. 3(c) shows that when a static pressure is applied, fluorescence is observed over the full thickness of the vascular wall under the same conditions of ultrasonic irradiation and surrounding medium, which indicates that delivery of the gene to deep parts is facilitated. The inventors' other experiments have also demonstrated that the delivery-facilitating effect is produced when the pressure is increased by only several percent. The result of the experiments shows the efficacy of the delivery system according to the present invention.

When the ultrasonic irradiation sequence preset for the delivery of a drug to the subject 6 is completed, the controller 18 sends, to the driver 17, a control signal to stop the drive, so that the transmission of the ultrasonic wave U from the ultrasonic transducer 9 is stopped. The controller 18 also outputs a drive stop signal, for example, to the pressure pump 12, so that the drive of the pressure pump 12 is stopped, which reduces the pressure in the airtight pressure container 1. The controller 18 may cause the display 20 to display instructions on how to release the pressure, such as to manually open the cover of the airtight pressure container 1 and remove the small vessel 3 from the airtight pressure container 1. In this case, the controller 18 may cause the display 20 to indicate that the pressure in the airtight pressure container 1 is reduced so that the small vessel 3 can be safely removed from the airtight pressure container 1. The operator may open the cover of the airtight pressure container 1 to remove the small vessel 3 from the airtight pressure container 1.

When the ultrasonic irradiation sequence preset for the delivery of a drug to the subject 6 is completed, the airtight pressure container 1 in the state at the end of the ultrasonic irradiation sequence is transferred to the medical diagnostic imaging apparatus 19 including a molecular imaging device such as a PET device. The PET image or the like of the subject 6 is produced using the medical diagnostic imaging apparatus 19.

The controller 18 receives the data of the PET image or the like of the subject 6 from the medical diagnostic imaging apparatus 19 and causes the display 20 to display the diagnostic imaging information of the subject 6 and the state of delivery of a drug into the subject 6. This allows the state of delivery of a drug into the subject 6 to be checked.

As a result of the check, if the delivery of the drug into the subject 6 is insufficient, the ultrasonic irradiation sequence may be repeated for the delivery of the drug to the subject 6.

The microbubbles used in the delivery of the drug with the ultrasonic wave U are highly sensitive to detection by ultrasonic diagnostic apparatus. Therefore, an ultrasonic diagnostic probe of an ultrasonic diagnostic apparatus is previously placed in the applicator 4 provided with the ultrasonic transducer 9. In such a system, the ultrasonic diagnostic apparatus transmits an ultrasonic wave from the ultrasonic diagnostic probe to the subject 6 in the small vessel 3, and the reflected wave is detected so that the concentration and reach of the microbubbles with respect to the subject 6 in the small vessel 3, especially the concentration and reach of the microbubbles in the target region of the subject 6 can be checked using the ultrasonic image.

After the concentration and reach of the microbubbles are checked, a static pressure is applied to the subject 6, while the ultrasonic wave U is irradiated, so that the delivery of the drug to the subject 6 is facilitated by a microjet that is generated when the microbubbles collapse (the sonoporation phenomenon). In the case where the ultrasonic diagnostic probe of the ultrasonic diagnostic apparatus is previously placed in the applicator, the effect of the delivery of the drug to the subject 6 can be checked with the ultrasonic image produced by the ultrasonic diagnostic apparatus.

Specifically, the ultrasonic wave U is irradiated, while the effect of delivery of the drug to the subject 6 is checked with the ultrasonic image produced by the ultrasonic diagnostic apparatus based on the very high sensitivity of the ultrasonic wave U to bubbles, so that the drug can be more effectively delivered, for example, at a time when a contrast medium is accumulated in tumor tissues of the subject 6. This allows a significant improvement in the therapeutic effect and a reduction in a dose of the drug.

The effect of delivery of the drug with the ultrasonic wave U is higher with a continuous wave than with a pulse wave. In addition, the inventors have already demonstrated that when the frequency of the ultrasonic wave U or the like is changed, the effect of delivery of the drug is further enhanced. Therefore, at the time of imaging, the distribution of bubbles may be imaged under low MI irradiation incapable of breaking the bubbles, and then the low MI irradiation may be changed to high MI continuous irradiation in the process of irradiating the therapeutic ultrasonic wave, so that delivery and treatment can be more efficiently performed than in the case where the irradiation with the pulse wave is maintained.

In the first embodiment described above, the subject 6 is placed in the airtight pressure container 1 whose inside is kept at a static pressure, and the subject 6 is irradiated with an ultrasonic wave, so that a drug is delivered to the subject 6. This process increases the effect of delivery of the drug to deep tissue parts of the subject 6 and more effectively facilitates the delivery of the drug, when the subject 6 such as an isolated organ for transplantation or a small animal accommodated in the small vessel 3 is treated by irradiation of the ultrasonic wave U for delivery of the drug, so that the drug can be more reliably delivered to the subject 6. According to the embodiment, a new ultrasonic local drug delivery system can be achieved which is useful for gene therapy, drug delivery treatment and so on.

Since the ultrasonic transducer 9 is configured to focus the ultrasonic wave U on the focal region S, the ultrasonic wave U can be irradiated to any target region of the subject 6. Therefore, drugs can be reliably delivered to living local parts of the subject 6.

Next, a second embodiment of the present invention is described below with reference to the drawings.

FIG. 4 is a schematic diagram showing an ultrasonic drug delivery apparatus, in which an airtight pressure container 30 is cylindrically shaped. The upper portion of the airtight pressure container 30 is provided with an openable cover 31. When the cover 31 is closed, the airtight pressure container 30 becomes airtight.

The airtight pressure container 30 has a cylindrical inner wall, and a plurality of ultrasonic transducers (a group of ultrasonic transducers) 32 are arranged at specific intervals along the circumference of the inner wall. The plurality of ultrasonic transducers 32 are integrally provided on the airtight pressure container 30. For example, the ultrasonic transducers 32 are in the form of rectangles of the same size and each emit an ultrasonic wave with a frequency of 100 kHz to 10 MHz. The arrangement interval between the ultrasonic transducers 32 and their size may vary depending on the size of the subject 34 or the like.

A solution 33, for example, which includes a cell suspension and microbubbles mixed therein, is held in the airtight pressure container 30, and a subject (a sample to which delivery is to be made) 34 such as an isolated organ for transplantation or a small animal is immersed in the solution 33.

The airtight pressure container 30 is connected to a pressure pump 36 through a pressure tube 35 and provided with a pressure sensor 37. The pressure pump 36 placed outside the airtight pressures container 30 injects a gas such as oxygen or air into the airtight pressure container 30 to control the pressure in the airtight pressure container 30. The pressure sensor 37 placed outside the airtight pressure container 30 detects the pressure in the airtight pressure container 30 and outputs a pressure detection signal.

A plurality of drivers 38 are provided outside the airtight pressure container 30 and each outputs a drive signal to each ultrasonic transducer 32 so that each ultrasonic transducer 32 is driven typically at a frequency of 100 kHz to 10 MHz to generate an ultrasonic wave U. The drivers 38 are connected to the ultrasonic transducers 32 through an airtight cable of the airtight pressure container 30 such that the drive signal output from each driver 38 is sent to each ultrasonic transducer 32.

A controller 39 outputs a drive signal to the pressure pump 36 to drive the pressure pump 36. The controller 39 also inputs the pressure detection signal output from the pressure sensor 37 to control the pressure in the airtight pressure container 30 at a constant positive static pressure, for example, a specific static pressure in the range of 1.05 to 3 atmospheres.

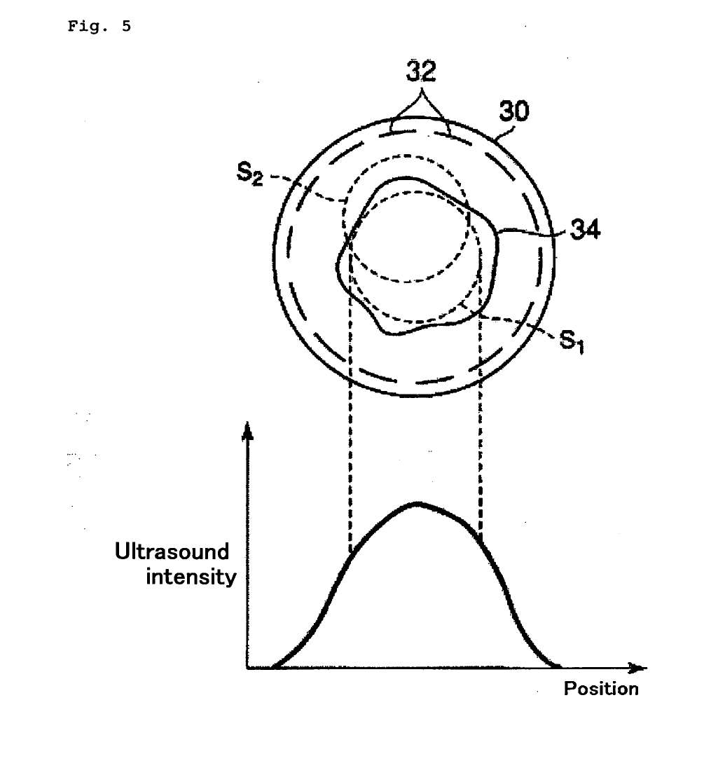

While the controller 39 keeps the inside of the airtight pressure container 30 at a static pressure, it sends a drive control signal to each driver 38 so that each ultrasonic transducer 32 is driven typically at a frequency of 100 kHz to 10 MHz to generate the ultrasonic wave U. The controller 39 sends, to each driver 38, each set of drive control signals to uniformly irradiate the ultrasonic wave throughout the subject 34 (such as signals to control the timing of oscillation of each ultrasonic transducer 32, the oscillating frequency, the phase, and the waveform in the process of oscillation of each ultrasonic transducer 32). FIG. 5 shows a focal region S₁ of the ultrasonic wave applied to the subject 34 in the airtight pressure container 30. It indicates that the ultrasonic wave is uniformly irradiated throughout the subject 34.

The controller 39 controls each set of drive control signals such as signals to control the timing of oscillation of each ultrasonic transducer 32, the oscillating frequency, the phase, and the waveform in the process of oscillation of each ultrasonic transducer 32. Therefore, the controller 39 can control the focal region of the ultrasonic wave U (for example, the focal region S₂ as shown in FIG. 5) and move it to the desired target region of the subject 34. When the target region is larger than the focal region S₂, the controller 39 can also move the region S₂ such that the target region can entirely and evenly irradiated.

Next, a description is given of the operation to facilitate delivery of the drug in the apparatus configured as described above.

A solution 33, for example, which includes a cell suspension and microbubbles mixed therein, is held in the airtight pressure container 30, and a subject (a sample to which delivery is to be made) 34 such as an isolated organ for transplantation or a small animal is immersed in the solution 33.

The controller 39 inputs a pressure detection signal output from the pressure sensor 37 and outputs a drive signal to the pressure pump 36 to keep the inside of the airtight pressure container 30 at a constant positive static pressure, for example, a specific static pressure in the range of 1.05 to 3 atmospheres.

The controller 39 outputs a drive signal to the pressure pump 36 to drive the pressure pump 36. The pressure pump 36 injects a gas such as oxygen or air into the airtight pressure container 30 through the pressure tube 35 to increase the pressure in the airtight pressure container 30. In this process, the pressure sensor 37 detects the pressure in the airtight pressure container 30 and outputs the pressure detection signal.

For example, when the pressure in the airtight pressure container 30 is kept at a static pressure of 1.05 atmospheres, the controller 39 sends, to each driver 38, each set of drive control signals to uniformly irradiate the ultrasonic wave throughout the subject 34 (such as signals to control the timing of oscillation of each ultrasonic transducer 32, the oscillating frequency, the phase, and the waveform in the process of oscillation of each ultrasonic transducer 32). The drivers 38 each output a drive signal to each ultrasonic transducer 32, so that each ultrasonic transducer 32 generates an ultrasonic wave typically with a frequency of 100 kHz to 10 MHz. In this process, the ultrasonic wave generated from each ultrasonic transducer 32 is uniformly irradiated throughout the subject 34 as shown in FIG. 5.

The controller 39 controls each set of drive control signals such as signals to control the timing of oscillation of each ultrasonic transducer 32, the oscillating frequency, the phase, and the waveform in the process of oscillation of each ultrasonic transducer 32, so that the focal region S₂ of the ultrasonic wave U can be controlled and moved to the desired target region of the subject 34 as shown in FIG. 5.

As described above, the ultrasonic wave U is uniformly irradiated to the subject 6, while a static pressure is applied to the subject 6. As a result, the interaction with the microbubbles is facilitated so that delivery of the drug to the subject 34 is facilitated by a microjet that is generated when the microbubbles collapse (the sonoporation phenomenon).

According to the second embodiment, a plurality of ultrasonic transducers 32 are integrally provided on the airtight pressure container 30, and a static pressure is applied to the subject 34 in the airtight pressure container 30, while ultrasonic waves are irradiated to the subject 34 from the plurality of ultrasonic transducers 32 for delivery of the drug to the subject 34. This allows effective delivery of drugs to the subject 34. In this case, each set of drive control signals such as signals to control the timing of oscillation of each ultrasonic transducer 32, the oscillating frequency, the phase, and the waveform in the process of oscillation of each ultrasonic transducer 32 are controlled so that the ultrasonic wave can be uniformly irradiated throughout the subject 34.

For example, when the subject 34 is an organ to be transplanted, a drug for suppressing rejection should be delivered throughout the organ to be transplanted, because many blood vessels exist over the organ to be transplanted. In the apparatus of this embodiment, the ultrasonic wave can be uniformly irradiated throughout the subject 34, and therefore, the drug can be delivered throughout the organ to be transplanted such that rejection of the organ to be transplanted can be suppressed.

The apparatus of this embodiment is also suitable for use in a therapy requiring urgent transfer and treatment, such as organ transplantation. For example, the apparatus of this embodiment having the plurality of ultrasonic transducers 32 integrally provided on the airtight pressure container 30 may be configured to be portable. Therefore, the apparatus of this embodiment may be used to deliver, to the subject 34, a drug for suppressing organ transplant rejection, while the organ to be transplanted is transported by air or the like. This allows a quick start of transplantation therapy, when the organ to be transplanted arrives at a hospital or the like where the therapy is to be conducted. It will be understood that the apparatus may also be used for ordinary delivery treatment other than the delivery of organ transplant rejection-suppressing drugs to the subject 34.

Additionally, each set of drive control signals such as signals to control the timing of oscillation of each ultrasonic transducer 32, the oscillating frequency, the phase, and the waveform in the process of oscillation of each ultrasonic transducer 32 are controlled so that the focal region S₂ of the ultrasonic wave U can be controlled and moved to the desired target region of the subject 34.

The second embodiment may also be modified as described below. For example, the ultrasonic transducer 32 maybe provided not only on the cylindrical inner wall of the airtight pressure container 30 but also on the bottom surface of the airtight pressure container 30, so that the ultrasonic wave. can be more uniformly irradiated throughout the subject 34.

Next, a third embodiment of the present invention is described below with reference to the drawings. The same parts are represented by the same reference numeral as shown in FIG. 2, and the detail description thereof will be omitted.

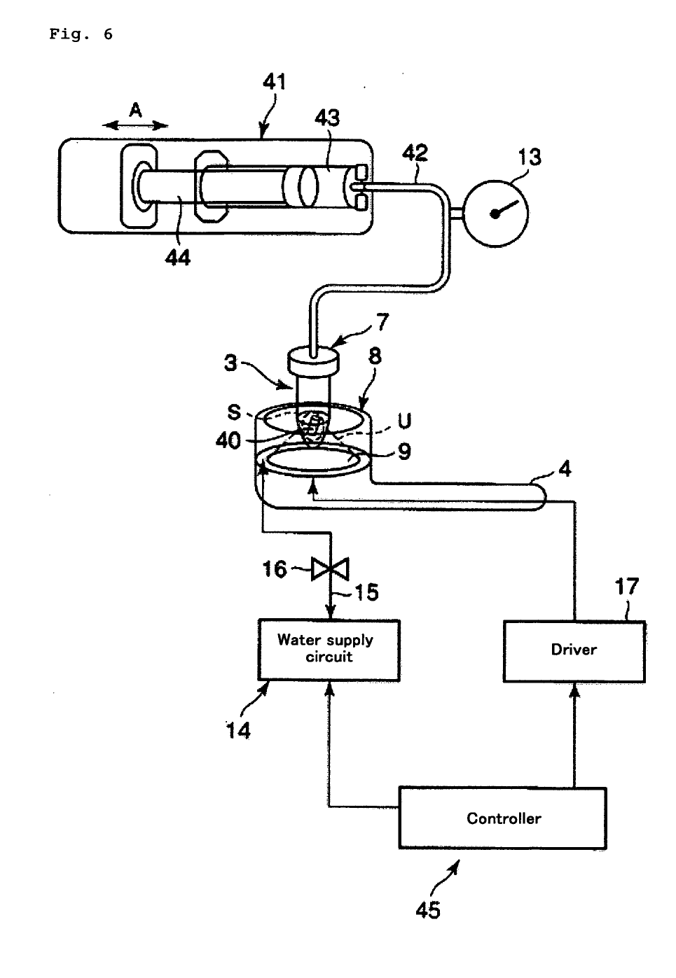

FIG. 6 is a schematic diagram showing an ultrasonic drug delivery apparatus. This apparatus is a simple pressurized drug delivery system for in vitro delivery of drugs to a small subject 40, which aims to enable delivery of drugs to the minute subject 40 such as a cell suspension and a blood vessel to be transplanted. For example, a standard container is used as the small vessel 3. The standard container 3 is for use in standard in vitro experiments and the like as mentioned above and, for example, includes a 15 ml tube (manufactured by Greiner). The standard container 3 is held in an applicator 4.

A syringe pressurizer 41 is connected to a pressure cap 7 of the standard container 3 through a pressure tube 42. The syringe pressurizer 41 injects a gas such as oxygen or air into the standard container 3 through the pressure tube 42 to control the pressure in the standard container 3. The syringe pressurizer 41 includes a compressing chamber 43 and a cylinder 44 provided slidably in the directions of an arrow A in the compressing chamber 43. The inner space of the compressing chamber 43 is compressed by the movement of the cylinder 44 so that a gas such as oxygen or air is supplied to the standard container 3. In the syringe pressurizer 41, the cylinder 44 may be automatically or manually allowed to slide. A pressure sensor 13 is attached to the pressure tube 42.

A controller 45 outputs a drive signal to the syringe pressurizer 41 to drive the syringe pressurizer 41. The controller 45 also inputs a pressure detection signal output from the pressure sensor 13 to control the pressure in the standard container 3 at a constant positive static pressure, for example, a specific static pressure in the range of 1.05 to 3 atmospheres.

While the controller 45 keeps the inside of the standard container 3 at a static pressure, it sends a drive control signal to a driver 17 so that an ultrasonic transducer 9 is driven typically at a frequency of 100 kHz to 10 MHz to generate an ultrasonic wave U.

Next, a description is given of the operation to facilitate delivery of a drug in the apparatus configured as described above.

The controller 45 sends an opening and closing control signal to the valve 16 to open the valve 16. The water supply circuit 14 then supplies water 10 to a housing 8 or a water bag through a water supply piping 15. When the housing 8 or the water bag is filled with water 10, the controller 45 sends an opening and closing control signal to the valve 16 to close the valve 16. Therefore, backflow of water from the housing 8 or the water bag to the water supply circuit 14 is prevented.

The standard container 3 contains a solution 5, for example, which includes a cell suspension and microbubbles mixed therein. The minute subject 40 such as a cell suspension or a blood vessel to be transplanted is immersed in the solution 5. The standard container 3 is sealed with the pressure cap 7 so that the inside of the standard container 3 is kept in an airtight state. The standard container 3 is held such that the subject 40 is placed in the focal region S of the ultrasonic wave U emitted from the ultrasonic transducer 9, namely such that the subject 40 is placed on a plane where the energy of the ultrasonic wave U emitted from the ultrasonic transducer 9 is irradiated.

The controller 45 outputs a drive signal to the syringe pressurizer 41 to drive the cylinder 44, so that the syringe pressurizer 41 injects a gas such as oxygen or air into the standard container 3 through the pressure tube 42 to increase the pressure in the standard container 3. In this process, the pressure sensor 13 detects the pressure in the standard container 3 and outputs a pressure detection signal.

The controller 45 inputs the pressure detection signal output from the pressure sensor 13 and outputs a drive signal to the syringe pressurizer 41 to keep the inside of the standard container 3 at a constant positive static pressure, for example, a specific static pressure in the range of 1.05 to 3 atmospheres. In the syringe pressurizer 41, the cylinder 44 is allowed to slide in the direction of the arrow A to compress the inner space of the compressing chamber 43 so that a gas such as oxygen or air is supplied to the standard container 3 through the pressure tube 42. In this process, the pressure in the standard container 3 increases. Alternatively, in the syringe pressurizer 41, the cylinder 44 may be manually allowed to slide in the direction of the arrow A to compress the inner space of the compressing chamber 43 so that a gas such as oxygen or air may be supplied to the standard container 3 through the pressure tube 42.

For example, when the inside of the standard container 3 is kept at a static pressure of 1.05 atmospheres, the controller 45 sends a control signal to the driver 17 to start the drive, so that the ultrasonic transducer 9 generates an ultrasonic wave U, for example, with a frequency of 100 kHz to 10 MHz. Since the standard container 3 is held such that the subject 40 is placed on the plane where the energy of the ultrasonic wave U emitted from the ultrasonic transducer 9 is irradiated, the subject 40 is irradiated with the ultrasonic wave U emitted from the ultrasonic transducer 9.

As described above, the ultrasonic wave U is irradiated to the subject 40, while a static pressure is applied to the subject 40. As a result, the interaction with the microbubbles is facilitated so that delivery of a drug to the subject 40 is facilitated by a microjet that is generated when the microbubbles collapse (the sonoporation phenomenon).

According to the third embodiment, the standard container 3 contains the solution 5, for example, which includes a cell suspension and microbubbles mixed therein, and the minute subject 40 such as a cell suspension or a blood vessel to be transplanted is immersed in the solution 5. While the inside of the standard container 3 is kept at a static pressure with the syringe pressurizer 41, the ultrasonic wave U is irradiated to the subject 40 in the standard container 3. These features achieve a simple pressurized drug delivery system for in vitro delivery of drugs to the small subject 40, which aims to enable delivery of drugs to the minute subject 40 such as a cell suspension and a blood vessel to be transplanted.

The embodiments described above are not intended to limit the scope of the present invention, and any modifications such as those described below are possible.

For example, also in each of the second and third embodiments, the airtight pressure container 30 shown in FIG. 4 or the standard container 3 shown in FIG. 6 may be moved to the medical diagnostic imaging apparatus 19 equipped with a molecular imaging device such as a PET device, after the delivery of the drug to the subject 6 is completed. PET images or the like of the subject 34 or 40 may be produced using the medical diagnostic imaging apparatus 19. The state of delivery of the drug into the subject 34 or 40 may be checked using the images.

As a result of the check, if the delivery of the drug into the subject 34 or 40 is insufficient, the ultrasonic irradiation sequence described above may be performed again for the delivery of the drug to the subject 34 or 40.

An ultrasonic diagnostic probe of an ultrasonic diagnostic apparatus may be previously placed in the airtight pressure container 30 shown in FIG. 4 or the applicator 4 shown in FIG. 6, so that the concentration and reach of the microbubbles in the subject 34 or 40 may be checked using ultrasonic images.

1-28. (canceled)

29. An ultrasonic drug delivery apparatus, comprising:
a static pressure application unit that applies a static pressure to a subject, wherein the static pressure application unit comprises a pressure container that accommodates the subject, a pressurizing mechanism that pressurizes the inside of the pressure container so as to apply the static pressure to the subject, and a pressure sensor that detects the pressure applied to the inside of the pressure container;
an ultrasonic wave application unit that applies an ultrasonic wave to the subject;
a drive unit that is provided outside the pressure container to drive at least one ultrasonic transducer; and
an airtight cable that connects the drive unit to the pressure container, wherein
a drug is delivered to the subject, while the static pressure and the ultrasonic wave are applied to the subject.

30. An ultrasonic drug delivery apparatus, comprising:
a static pressure application unit that applies a static pressure to a subject and comprises a pressure container; and
an ultrasonic wave application unit that applies an ultrasonic wave to the subject, wherein
a drug is delivered to the subject, while the static pressure and the ultrasonic wave are applied to the subject,
the pressure container is a standard container for irradiation of the ultrasonic wave to the subject,
the ultrasonic wave application unit has an ultrasonic transducer to emit the ultrasonic wave and accommodates an acoustic medium that is provided with the ultrasonic transducer and acoustically connects the ultrasonic transducer to the standard container, and
the ultrasonic wave application unit has a holding part that holds the standard container such that the standard container coincides with a region on which the irradiation of the ultrasonic wave emitted from the ultrasonic transducer is focused.

31. An ultrasonic drug delivery apparatus, comprising:
a static pressure application unit that applies a static pressure to a subject and comprises a pressure container; and
an ultrasonic wave application unit that applies an ultrasonic wave to the subject, wherein
a drug is delivered to the subject, while the static pressure and the ultrasonic wave are applied to the subject,
the pressure container is cylindrical,
the ultrasonic wave application unit comprises a plurality of ultrasonic transducers to emit the ultrasonic wave, and
the plurality of ultrasonic transducers are arranged at least on a cylindrical inner wall of the pressure container.

32. The ultrasonic drug delivery apparatus according to claim 31, wherein the plurality of ultrasonic transducers each emit the ultrasonic wave such that the ultrasonic wave is uniformly irradiated throughout the subject.

33. The ultrasonic drug delivery apparatus according to claim 31, further including a drive control unit that controls the drive of the plurality of ultrasonic transducers by at least phase control.

34. An ultrasonic drug delivery apparatus, comprising:
a static pressure application unit that applies a static pressure to a subject and comprises a pressure container and a pressurizing mechanism; and
an ultrasonic wave application unit that applies an ultrasonic wave to the subject, wherein
a drug is delivered to the subject, while the static pressure and the ultrasonic wave are applied to the subject,
the pressure container is a standard container for irradiation of the ultrasonic wave to the subject,
the pressurizing mechanism is a syringe pressurizer and pressurizes the inside of the standard container such that the static pressure is applied to the subject, and
the ultrasonic wave application unit comprises an ultrasonic transducer to emit the ultrasonic wave and applies the ultrasonic wave from the outside of the standard container to the subject.

35. An ultrasonic drug delivery apparatus, comprising:
a static pressure application unit that applies a static pressure to a subject and comprises a pressure container; and
an ultrasonic wave application unit that applies an ultrasonic wave to the subject, wherein
a drug is delivered to the subject, while the static pressure and the ultrasonic wave are applied to the subject, and
the pressure container is made of a material that allows delivery of the drug to the subject to be checked with a molecular imaging device.

36. The ultrasonic drug delivery apparatus according to claim 35, wherein the material of which the pressure container is made comprises an optically-transparent material that allows fluorescent imaging.

37. The ultrasonic drug delivery apparatus according to claim 35, wherein the material of which the pressure container is made is transparent to radioactive rays or X-rays.

38. The ultrasonic drug delivery apparatus according to claim 35, wherein the material of which the pressure container is made allows magnetic resonance imaging.

39. A medical diagnostic imaging apparatus, comprising the ultrasonic drug delivery apparatus according to claim 29.

40. The medical diagnostic imaging apparatus according to claim 39, further comprising an ultrasonic transducer that is for producing an ultrasonic image of the subject and independently placed in the pressure container, wherein the ultrasonic image of the subject is output and displayed, while the drug is delivered to the subject under application of the static pressure and the ultrasonic wave.

41. The medical diagnostic imaging apparatus according to claim 39, wherein the ultrasonic transducer for producing the ultrasonic image of the subject also serves to apply the ultrasonic wave to the subject for delivery of the drug to the subject.

42. The medical diagnostic imaging apparatus according to claim 39, further comprising a device for positron emission tomography (PET), magnetic resonance imaging (MRI) or X-ray computed tomography (CT).
